(19) United States
(12) Patent Application Publication
(10) Pub. No.: US 2021/0272662 A1
(43) Pub. Date: Sep. 2, 2021

(54) APPLICATION, SYSTEM, AND METHOD FOR A COMPUTER IMPLEMENTED MEDICAL ELECTRONIC RECORD MANAGEMENT SYSTEM

(71) Applicant: Lawrence German, San Juan, PR (US)

(72) Inventors: Stan Gutkin, Mt Arlington, NJ (US); Abha Chalpe Ghosh, Pune (IN); Kaouk Ahmad Sami, Odessa (UA)

(21) Appl. No.: 17/186,520

(22) Filed: Feb. 26, 2021

Related U.S. Application Data
(60) Provisional application No. 62/982,212, filed on Feb. 27, 2020.

(51) Int. Cl. G16H 10/60; G16H 40/20; G06F 16/95

(52) U.S. Cl. CPC G16H 10/60; G06F 16/95; G16H 40/20

(57) ABSTRACT

Computer implemented methods and associated systems for decreasing incidence of an uncontrolled medical attribute of a plurality of patients in a patient population thereby identifying risk and reducing incidence of hospitalization and patient readmission are disclosed.

### CROSS-REFERENCE TO RELATED APPLICATIONS

This application claims the benefit of U.S. Provisional Application No. 62/982,212, filed on Feb. 27, 2020 and entitled, “APPLICATION, SYSTEM, AND METHOD FOR A COMPUTER IMPLEMENTED MEDICAL ELECTRONIC RECORD MANAGEMENT SYSTEM,” the subject matter of which is hereby incorporated by reference.

### STATEMENT REGARDING FEDERALLY SPONSORED RESEARCH OR DEVELOPMENT

Not applicable.

### INCORPORATION BY REFERENCE OF MATERIAL SUBMITTED ON A COMPACT DISC

Not applicable.

### TECHNICAL FIELD

The present invention relates to the field of record access and management, and more specifically to the field of systems and methods for communication between healthcare professionals and healthcare consumers (patients) for managing a healthcare consumer's health data.

### BACKGROUND

The Centers for Medicare and Medicaid Services (CMS) recognizes Chronic Care Management (CCM) as a critical component of primary care that contributes to better health and care for individuals. CCM encompasses the oversight and education activities conducted by health care professionals to help patients with chronic diseases and health conditions such as diabetes, high blood pressure, systemic lupus erythematosus, multiple sclerosis, and sleep apnea learn to understand their condition and live successfully with it. This term is equivalent to disease management for chronic conditions. The work involves motivating patients to persist in necessary therapies and interventions and helping them to achieve an ongoing, reasonable quality of life.

Although deploying a chronic care management program is a great opportunity for providers to enhance their relationship with their patients while also generating a new revenue stream for the practice, for many provider organizations, the significant staffing and technology investments required for CCM is labor intensive and cost prohibitive. For example, necessary interventions can require input from multiple specialists that may not usually work together, and to be effective, they require close, careful coordination. However, the lack of ability to uniformly coordinate across multiple settings, providers and treatments of chronic illness care outweigh the benefits of adopting a CCM program.

Additionally, the regulatory guidelines of CCM tracking and reporting, along with the expertise needed to aggregate and analyze patient data discourage many providers, including those at large practices, from establishing a CCM program. For example, among the requirements outlined by CMS for CCM is a 20-minute minimum of non-face-to-face care coordination and management with each patient per month. Due to the strict criteria regarding the amount of time spent with each patient, many providers find that they do not have a systematic way of recording these activities for tracking purposes, on top of providing prompt, effective care to their patients.

Therefore, a need exists to improve over the prior art and more particularly, for a medical electronic record management system to improve chronic care management, reduce patient risk, and decrease hospital readmission.

### SUMMARY

A system and method for decreasing incidence of an uncontrolled medical attribute of a plurality of patients in a patient population is disclosed. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

In one embodiment, a computer implemented method executed by a first computing device for decreasing incidence of an uncontrolled medical attribute of a plurality of patients in a patient population thereby identifying risk and reducing incidence of hospitalization and patient readmission is disclosed. The method comprises (a) generating a patient record for each of the plurality of patients, (b) storing, in an attached database and in the patient record for each of the plurality of patients, (i) a unique patient identifier for each patient, (ii) patient identifying information and (iii) patient medical data, wherein the patient medical data comprises measurements related to at least one medical attribute of the patient, (c) generating a coordinator record for each of a plurality of coordinator devices, wherein each coordinator device is associated with a coordinator, (d) storing, in the attached database, in the coordinator record, wherein the coordinator record comprises a unique coordinator identifier associated with the particular coordinator, (e) generating first information associated with a particular patient from the plurality of patients, wherein the first information comprises (i) the unique patient identifier of the particular patient and (ii) first custom webpage information for displaying a first custom webpage comprising a plurality of regions for receiving coordinator input data from a particular coordinator computing device, (f) transmitting, to the particular coordinator computing device, the first information to have the first custom webpage displayed on the particular coordinator computing device, (g) receiving, from the particular coordinator computing device, the coordinator input data, wherein the coordinator input data comprises (i) the unique patient identifier associated with the first custom webpage, (ii) the unique coordinator identifier associated with the particular coordinator computing device and (iii) at least an amount of time the particular coordinator computing device interacted with each of the plurality of regions of the first custom webpage, (h) storing, in the particular coordinator record, the coordinator input data, (i) generating second information associated with the particular coordinator computing device, wherein the second information comprises (i) the unique coordinator identifier of the particular coordinator; and, (ii) second custom webpage information for displaying a second custom webpage on a supervisor computing device, wherein the second custom webpage information comprises the coordinator input data associated with the particular coordinator, (j) transmitting, to the supervisor computing device, the second information to be displayed on the supervisor computing device, wherein the second custom webpage comprises a first graphical representation of a timeline indicating, based on the coordinator input data received from the particular coordinator computing device, (i) when the particular coordinator computing device was engaging with regions on the first custom webpage over a first period of time and (ii) when the particular coordinator computing device was not engaging with the regions on the first custom webpage over the first period of time.

In one embodiment, a system for decreasing incidence of an uncontrolled medical attribute of a plurality of patients in a patient population comprises: (a) a first server comprising at least one processor, (b) a particular coordinator computing device associated with a particular coordinator, and (c) a supervisor computing device associated with a particular supervisor. In one embodiment, the system comprises a provider computing device.

Additional aspects of the disclosed embodiment will be set forth in part in the description which follows, and in part will be obvious from the description, or may be learned by practice of the disclosed embodiments. The aspects of the disclosed embodiments will be realized and attained by means of the elements and combinations particularly pointed out in the appended claims. It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute part of this specification, illustrate embodiments of the invention and together with the description, serve to explain the principles of the disclosed embodiments. The embodiments illustrated herein are presently preferred, it being understood, however, that the invention is not limited to the precise arrangements and instrumentalities shown, wherein:

FIG. 1a is a diagram of an operating environment according to an example embodiment that supports a system for decreasing incidence of an uncontrolled medical attribute of a plurality of patients in a patient population thereby identifying risk and reducing incidence of hospitalization and patient readmission;

FIG. 1b is hierarchy illustrating a management structure of the relevant entities depicted in FIG. 1a;

FIG. 2a is a block-flow diagram of a method for decreasing incidence of an uncontrolled medical attribute of a plurality of patient, depicting the steps up to off-screen connector A;

FIG. 2b is a block-flow diagram of a method for decreasing incidence of an uncontrolled medical attribute of a plurality of patient, depicting the steps after off-screen connector A from FIG. 2a and up to off-screen connector B;

FIG. 2c is a block-flow diagram of a method for decreasing incidence of an uncontrolled medical attribute of a plurality of patient, depicting the steps up after off-screen connector B from FIG. 2b;

FIG. 3a is a diagram illustrating the flow of information according to a disclosed example embodiment;

FIG. 3b is a diagram illustrating the flow of information according to a disclosed example embodiment;

FIG. 4a is an embodiment of a custom webpage for entering coordinator input data that is sent to a coordinator and displayed on the coordinator's device;

FIG. 4b is an embodiment of a custom webpage comprising an incomplete medical care plan that is sent to a coordinator and displayed on the coordinator's computing device;

FIG. 4c is an embodiment of a custom webpage comprising a completed medical care plan that is sent to a coordinator and displayed on the coordinator's computing device;

FIG. 4d is an embodiment of a custom webpage for a coordinator comprising an action message sent by a server due in connection with a trigger event;

FIG. 5a is an embodiment of a custom webpage that comprises a timeline that illustrates the productivity of a particular coordinator;

FIG. 5b is an embodiment of a custom webpage that comprises parallel timeline that illustrates the productivity of at least two particular coordinators;

FIG. 5c is an embodiment of a custom webpage that shows a graphical representation of medical attribute data (blood pressure);

FIG. 5d is an embodiment of a custom webpage for a supervisor comprising an action message sent by the server due to a trigger event;

FIG. 5e is an embodiment of an action message sent to a patient via SMS and displayed on a patient's computing device; and

FIG. 6 is a block diagram of a system including an example computing device and other computing devices, according to an example embodiment.

### DETAILED DESCRIPTION

The following detailed description refers to the accompanying drawings. Whenever possible, the same reference numbers are used in the drawings and the following description to refer to the same or similar elements. While disclosed embodiments may be described, modifications, adaptations, and other implementations are possible. For example, substitutions, additions or modifications may be made to the elements illustrated in the drawings, and the methods described herein may be modified by substituting reordering or adding additional stages or components to the disclosed methods and devices. Accordingly, the following detailed description does not limit the disclosed embodiments. Instead, the proper scope of the disclosed embodiments is defined by the appended claims.

The disclosed embodiments improve upon the problems with the prior art by providing methods for providing healthcare services (e.g., CCM) to patients at a reduced cost. Moreover, the disclosed embodiments provide healthcare providers increased abilities to gauge risk in providing healthcare, such as the risk of readmittance for a particular condition. The disclosed embodiments additionally provide healthcare providers the ability to improve data sharing among the hierarchal ladder that is inherently present in a healthcare providing organization. Lastly, the disclosed embodiments increase the productivity of healthcare workers. For instance, the productivity of healthcare workers is increased due to providing methods for providing care to patients virtually. Furthermore, the productivity of each care coordinators, the overall productivity, and risks to productivity (e.g., understaffing, overstaffing) are easily audited by the supervisors and/or health care providers within the health care organization.

### i. Operating Environment and Participating Entities

As will become apparent from the following description, the disclosed systems and methods provide computer implemented (i.e., software) methods for providing medical care to a plurality of patients. The disclosed embodiments, generally speaking, are for decreasing incidence of an uncontrolled medical attribute of a plurality of patients in a patient population thereby identifying risk and reducing incidence of hospitalization and patient readmission. An uncontrolled medical attribute may be a particular condition or ailment, such as diabetes, hypertension, dementia, among many others. The disclosed embodiments provide health care providers the ability to manage the patient's medical attribute. For instance, coordinators and supervisors that work beneath a health care provider (e.g., a particular doctor or physician) are tasked with obtaining medical attribute data from the patient. The medical attribute data is stored electronically, and readily accessed by the health care provider. Moreover, the medical attribute data may be presented using unique interfaces that allow the health care provider to readily determine if intervention(s) are needed in the patient's care. In this way, health care providers can easily and effectively manage their patient's care. In this way, revenues for the health care provider may be increased while simultaneously lowering the risk of progression of the patient's ailment.

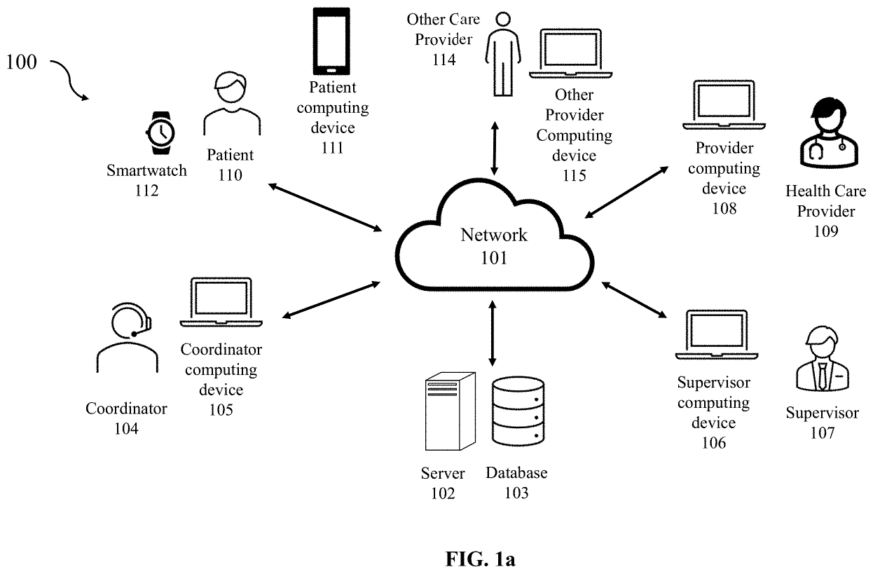

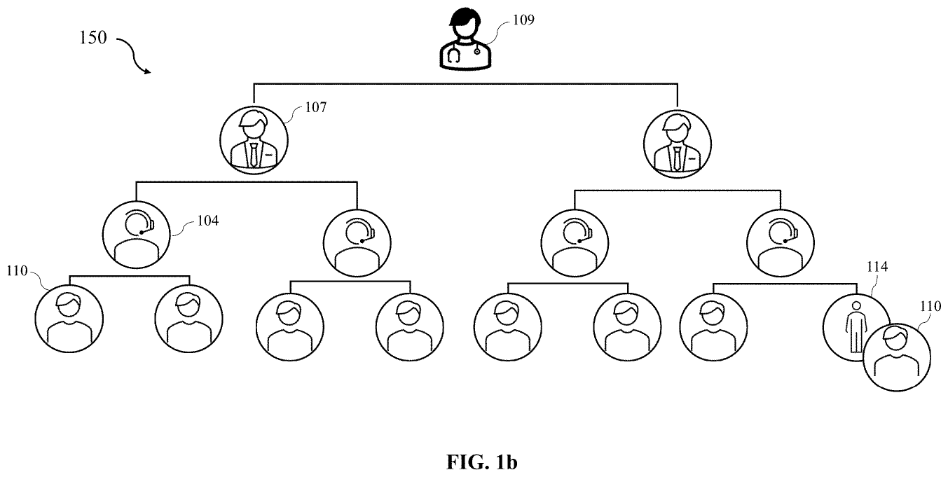

Referring now to FIGS. 1a-1b, an embodiment of an operating environment 100 (sometimes referred to herein as a system) is shown (FIG. 1a) and a hierarchy depicting a management structure 150 (FIG. b) is shown. As illustrated, the operating environment comprises the following entities: a first computing device 102 (depicted as a server), at least one coordinator 104, at least one supervisor 107, at least one health care provider 109, at least one patient 110, and optionally including at least one other care provider 114. As illustrated in FIG. 1b, the healthcare services of the present disclosure are managed in an exemplary hierarchy depicted by management structure 150. As illustrated by the exemplary management structure 150, the health care provider 109 directly manages at least one supervisor 107. The health care provider 109 may be, for instance, a particular doctor, a medical practice, or any other health care provider that is responsible for advising patients. The supervisors 107 directly manage at least one coordinator 104.

As will become more apparent from the foregoing description, the coordinator 104 is directly responsible for managing one or more patients 110. The tasks that are applicable for the coordinator 104 are generally speaking, specifically tailored to minimize the risk of progression of a particular ailment. By reducing the risk of progression, the risk of readmittance of a patient for their particular ailment is also significantly reduced. For instance, a coordinator 104 may manage a patient 110 who suffers from diabetes. Patient 110, who was recently admitted to the hospital for diabetes-induced neuropathy, poses a risk of readmittance if they do not take the steps to manage their diabetes. Patient 110 may be an indigent individual (e.g., elderly) and may require additional, outside help for managing their diabetes condition. In this way, the coordinator 104 may check-up on the patient 110 (e.g., by phone, or electronically such as by email, SMS message, etc.). The check-ups may include, for instance, having the patient 110 measure blood glucose levels to report to the coordinator 104, advising the patient 110 on dietary choices, reminding the patient 110 to exercise, and more. In sum, the coordinator may provide an additional layer of care between the health care provider 109 and the patient 110. The health care activities tasked to the coordinator generally relate to reducing risk in a patient (e.g., readmittance).

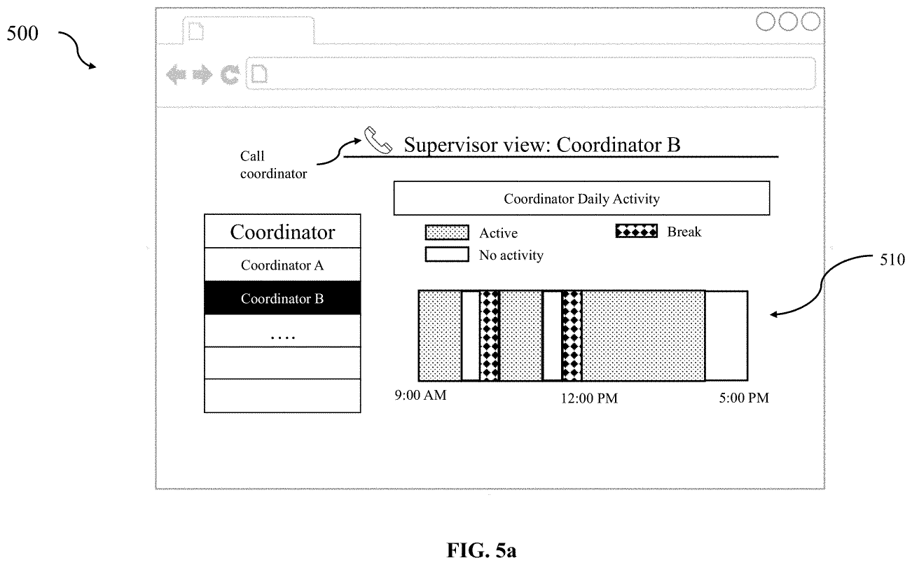

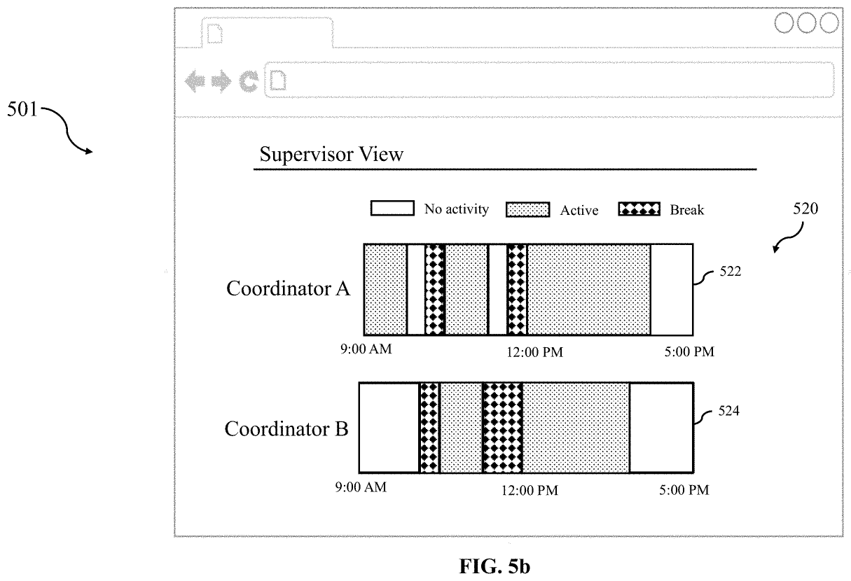

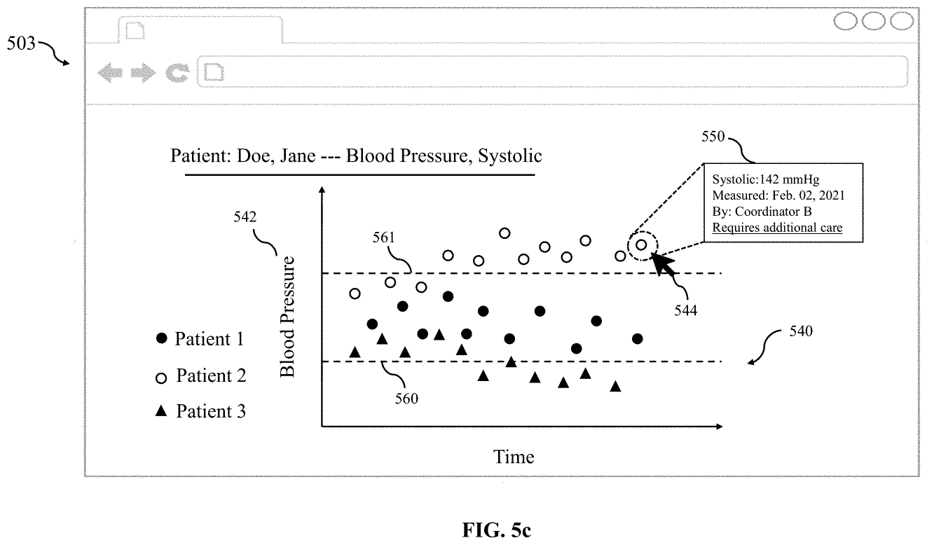

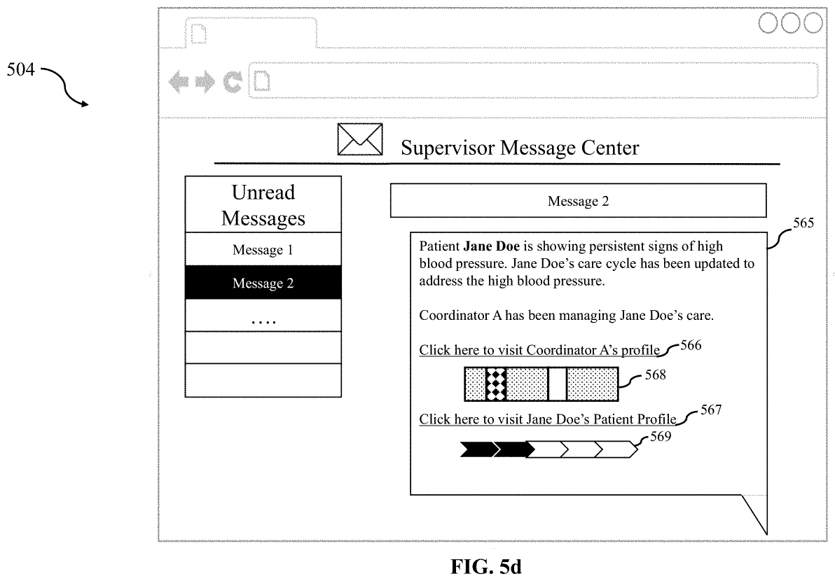

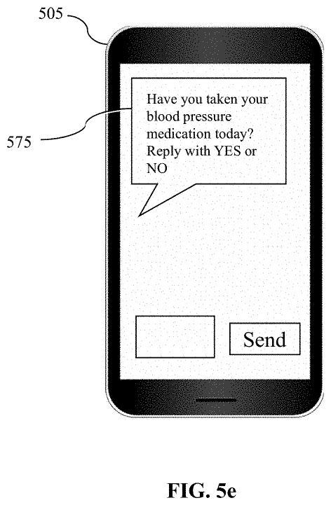

Supervisor 107 may act as an intermediary between health care provider 109 and coordinator 104. Supervisor 107 may be responsible for overseeing the workflow of the one or more coordinators 104. For instance, supervisor 107 may be responsible for the oversight of the workload of coordinators 104 (discussed in greater detail below). For instance, the oversight of coordinators may be related to productivity issues as well ensuring that access to healthcare meets certain predetermined standards. Productivity issues such as the coordinators having too much work (no coordinators are available at certain times of day), too little work, and/or not having an acceptable work ethic may be handled by supervisors using the systems and methods described herein. For instance, the supervisor may be able to readily envision productivity problems in their healthcare organization by reviewing parallel timelines of coordinator's work activity. In this regard, see FIG. 5b, discussed in greater detail below. While the illustrated embodiments depict a singular layer of supervisors, multiple layers of supervisors may be used. For instance, an upper-level supervisor may manage a plurality of lower-level supervisors, and the lower-level supervisors may directly manage a plurality of coordinators. Such multiple-level hierarchies, while not depicted, are within the spirit and scope of the invention.

The other care provider 114 may be an individual who can more directly aid in the care of patient 110. For instance, the other care provider 114 may be a nursing home attendant, an individual who is close to the patient (e.g., family member such as son/daughter, or other loved one), among others. The other care provider 114 may be an intermediary between the patient 110 and the coordinator 104, such as depicted in the lower right portion of FIG. 1b. In this way, tasks that the coordinator 104 is unable to accomplish by distant communication only (e.g., via phone, over the internet), such as measuring blood pressure, may be performed by the other care provider 114. In this way, the other care provider 114 may aid in the administration of health care to the patient 110. The other care provider 114 may participate using other provider computing device 115. For instance, the other care provider 114 may be sent a similar SMS message, such as the one depicted by FIG. 5e, to answer questions on behalf of the patient 110. The other provider computing device 115 may communicate with server 102, or any of the other devices (105, 106, 108, 111) over network 101 as shown by FIG. 1a.

The coordinator 104, supervisor 107, health care provider 109, and patient 110 are communicably connected via their respective computing devices (105, 106, 108, 111 and 112) via communications network 101. Communications network 101 may include one or more packet switched networks, such as the Internet, or any local area networks, wide area networks, enterprise private networks, cellular networks, phone networks, mobile communications networks, or any combination of the above. Communications sent over the network may be sent using any appropriate protocol. For instance, any communications described herein may be sent using Hypertext Transfer Protocol (“HTTP”) or Hypertext Transfer Protocol Secure (“HTTPS”). HTTPS is generally preferable due to increased security. Communications may also be sent via SMS, where the particular computing device is a remote computing device such as a cellular phone.

Computing devices (105, 106, 108 111) may be, for instance, remote computing devices such as a cellular telephone (e.g., IPHONE®, ANDROID®), tablet (e.g., IPAD®), smart phone or any other mobile device. Other computing devices, such as desktop computers, laptop computers, and gaming consoles may be used as well and are within the spirit and scope of the invention.

Patient 110 may also use a wearable device 112 such as an activity band (e.g., FITBIT®) or a smart watch (e.g., APPLE WATCH®). Wearable device 112 may record biometric data and may send the biometric data to server 102 to be stored in database 103 and inspected by one or more of the coordinator, supervisor, and health care provider. The biometric data may be, for instance, body temperature data, heart rate data, blood pressure data, activity (e.g., walking, exercise) data, sleep data, among others. Such biometric data may be leveraged to reduce risk in the patient 110. For instance, the disclosed methods and systems enable a health care provider to accumulate this biometric data, and to analyze it to determine if a patient's risk of progression of an ailment is increasing. A health care provider may also be able to determine if certain indicators of ailment progression are coupled. As a non-limiting example, a health care provider may see that a patient's blood pressure increase is coupled with a decrease in physical activity. The health care provider may decrease the patient's risk by tasking a coordinator to contact the patient to encourage them to exercise more.

Server 102 includes a software engine that delivers applications, data, program code and other information to networked devices (105, 106, 108 111, 112). The software engine of server 102 may perform other processes such as transferring multimedia data in a stream of packets that are interpreted and rendered by a software application as the packets arrive. FIG. 1a further shows that server 102 includes a database or repository 103, which may be a relational database comprising a Structured Query Language (SQL) database stored in a SQL server or a database that adheres to the NoSQL paradigm. The other computing devices (105, 106, 108 111, 112) may also each include databases (not illustrated).

### ii. Methods

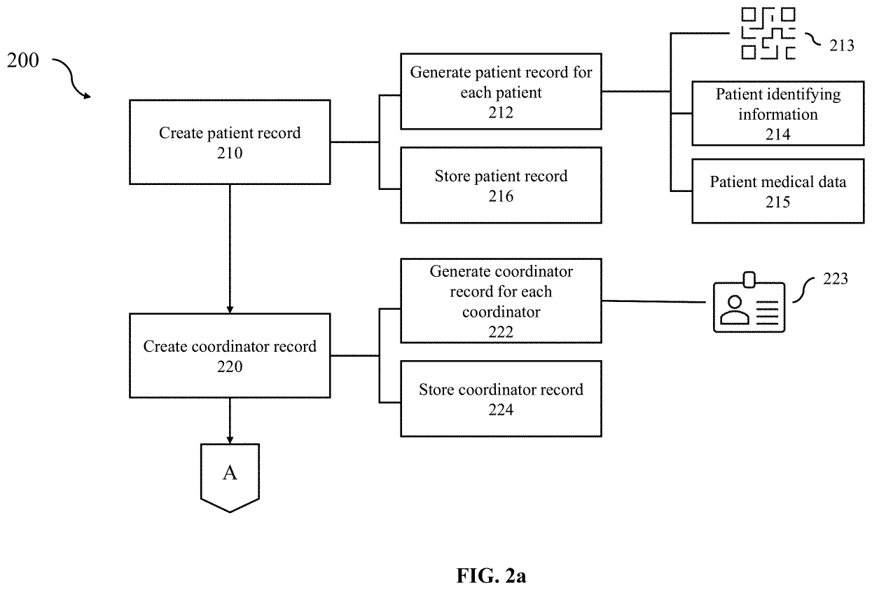

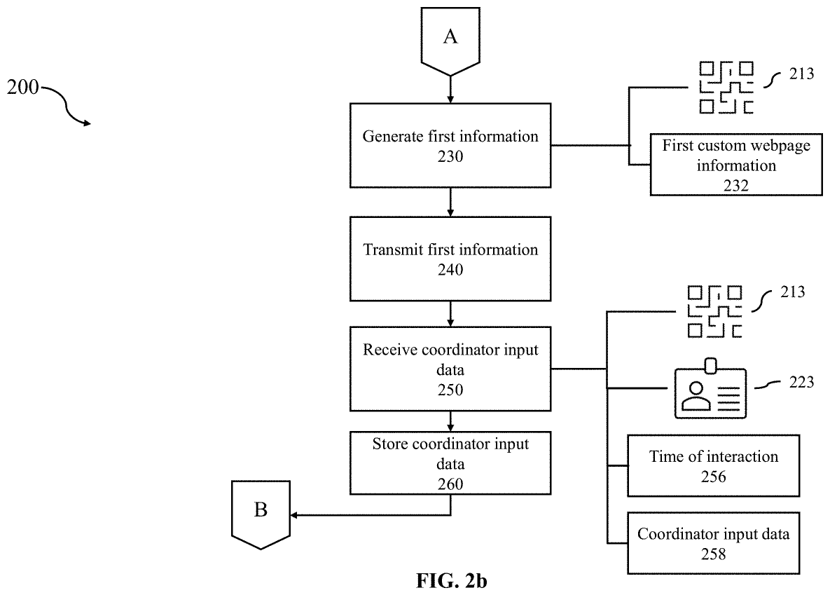

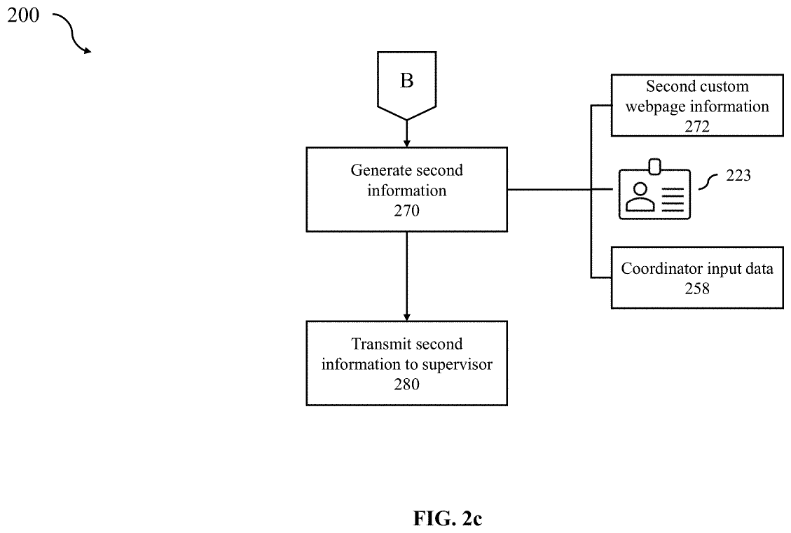

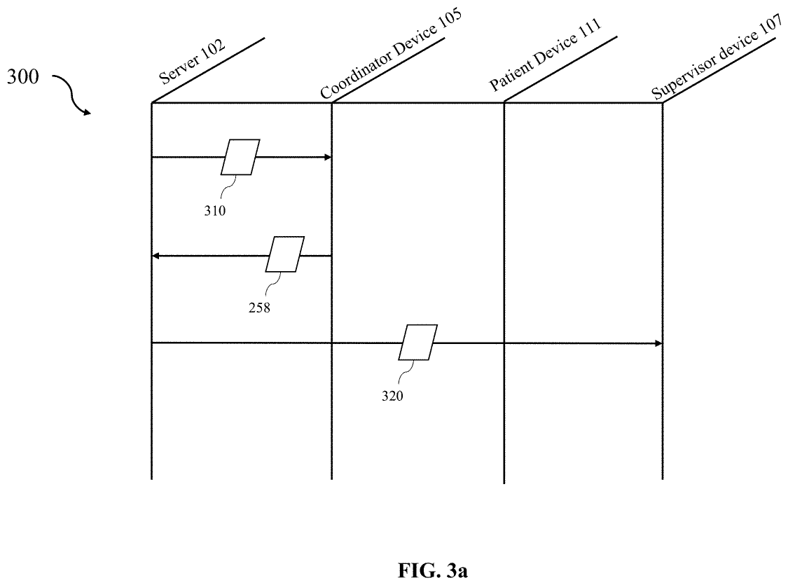

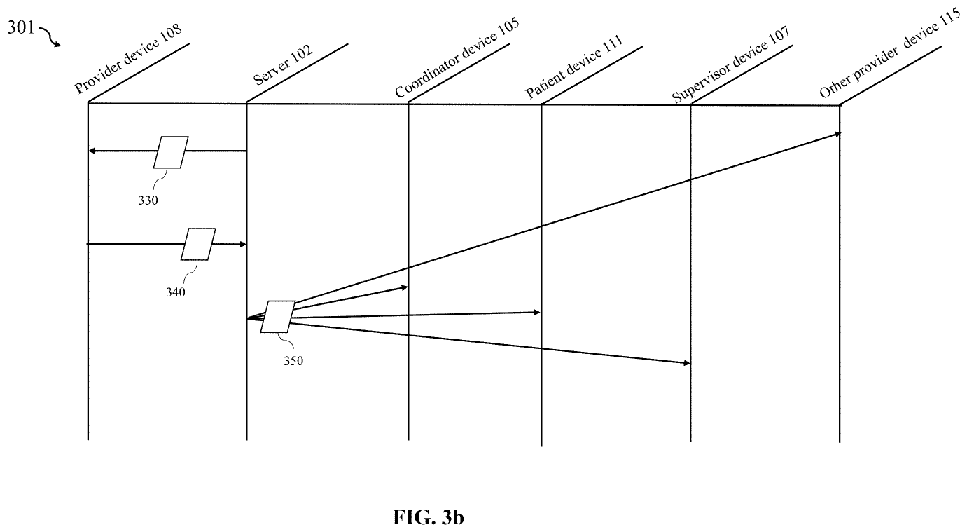

With reference to the figures now including FIGS. 2a-3b, a block-flow diagram of a method 200 for decreasing incidence of an uncontrolled medical attribute of a plurality of patients (FIGS. 2a-2c) and diagrams illustrating the flow of information 300, 301 (FIGS. 3a-3b) are shown. Method 200 is from the perspective of server 102. As illustrated, the method 200 comprises creating a patient record 210. The creating step 210 comprises generating a patient record 212 for each of the plurality of patients and subsequently storing the patient record 216 in, for instance, attached database 103. Each patient record for each patient 110 may comprise (i) a unique patient identifier 213 for each patient, (ii) patient identifying information 214 and (iii) patient medical data 215.

In one embodiment, prior to generating the patient record 212 for the particular patient, a method 200 comprises (not illustrated) receiving from the patient computing device at the direction of the patient, (i) the unique patient identifier of the particular patient, (ii) the patient identifying information of the particular patient and (iii) the patient medical data of the particular patient. In other words, in some embodiments the patient record is produced using data provided by the patient 110. The patient 110 may be provided a custom webpage having a user interface to provide this information, thus saving time of coordinator 104 and reducing costs to the health care provider 109. The patient 110 may provide this data, for instance, via a remote computing device such as a mobile phone. The patient's other care provider 114 may also aid the patient 110 in providing this data.

The unique patient identifier 213, as illustrated, is a quick response code (“QR code”). However, various types of unique identifiers may be used. For instance, a unique identifier may simply be a unique identification code, such as unique combinations of alphanumeric characters. Other unique identification codes may be, for instance, a bar code (e.g., codablock), an access code (e.g., a string of random numbers/letters of a specified length), an identification (“id”) tag, a hexadecimal code, a binary code, a bokode, a high-capacity color barcode (HCCB), and/or any other suitable machine-readable representation of data. A unique identifier may also be a government-issued identifier, such as a Medicare identification number, or a social security identification number, to name a few.

The patient identifying information 214 may be any combination of personal identifying information (“PII”), such as such as name, email address, cell phone number, home phone number, physical address, emergency contact information, among others. The patient identifying information 214 may also include PII for an other care provider 114. In one embodiment, the patient identifying information comprises at least one of a patient first name, a patient last name, a patient age, a patient email address, a patient unique identifier, a patient contact number, and a patient physical address.

Patient medical data 215 may comprise measurements related to at least one medical attribute of the patient. For instance, an attribute such as blood glucose may be measured and recorded in the patient record on a regular basis. Other exemplary attributes include sleep data, exercise data (e.g., daily steps), heart rate, blood pressure, and body temperature, among others. As a non-limiting example, these attributes may be measured directly by wearable device 112. Wearable device 112 may allow for continuous monitoring of the patient 110. In the event that the patient 110 prefers not to wear a wearable device, or that the relevant medical attribute is not measurable by a wearable device, a coordinator 104 may collect this data by contacting patient 110 (e.g., by phone call, by email, by SMS message, etc.). Other types of patient medical data that may be stored include communications between the particular coordinator computing device and the provider computing device, communications between the patient computing device and the particular coordinator computing device, patient medical history, patient prescription information, patient surgical information or any medical information related to the patient.

Similar to the patient 110, a record may be created and stored for each health care provider 109, each coordinator 104, and each supervisor 107. In this way, method 200 comprises a creating a coordinator record 220 step. The creating 220 may comprise generating a coordinator record 222 for each of a plurality of coordinator devices 105. Each coordinator device 105 is associated with a particular coordinator 104. The creating step 220 also comprises storing the coordinator record 224, such as in attached database 103. Generally speaking, the coordinator record comprises a unique coordinator identifier 223 associated with the particular coordinator 104. The coordinator identifier 223 is illustrated as an ID card of sorts. However, it is to be understood that the coordinator identifier 223 may be any unique identifier, such as any of the identifiers discussed above in relation to unique patient identifiers 213.

Method 200 further comprises generating first information 230. The first information 310 is generally associated with a particular patient 110 from the plurality of patients. The first information 310 may comprise (i) the unique patient identifier 213 of the particular patient 110 and (ii) first custom webpage information 232 for displaying a first custom webpage comprising a plurality of regions for receiving coordinator input data 258 from a particular coordinator computing device 105. The method 200 comprises transmitting 240 the first information 310 to the particular coordinator computing device. The transmitting step 240 may comprise transmitting via at least one of HTTP and HTTPS.

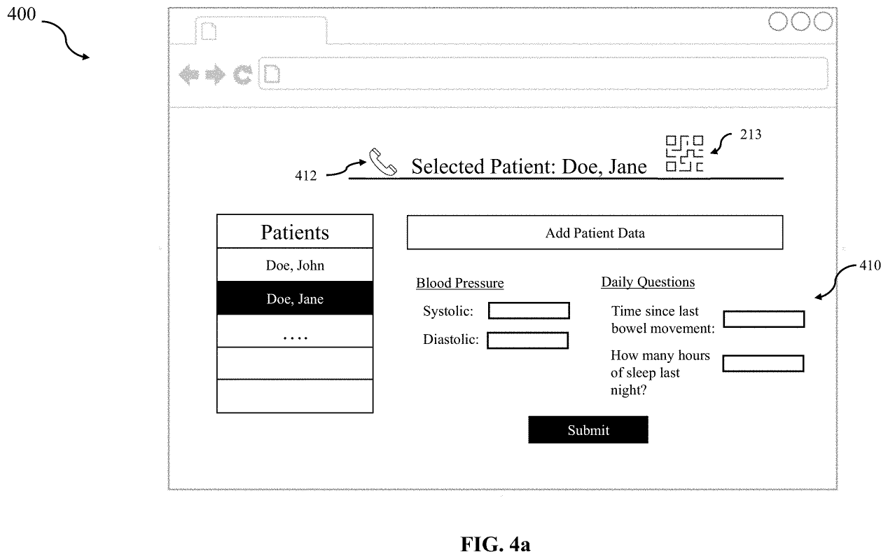

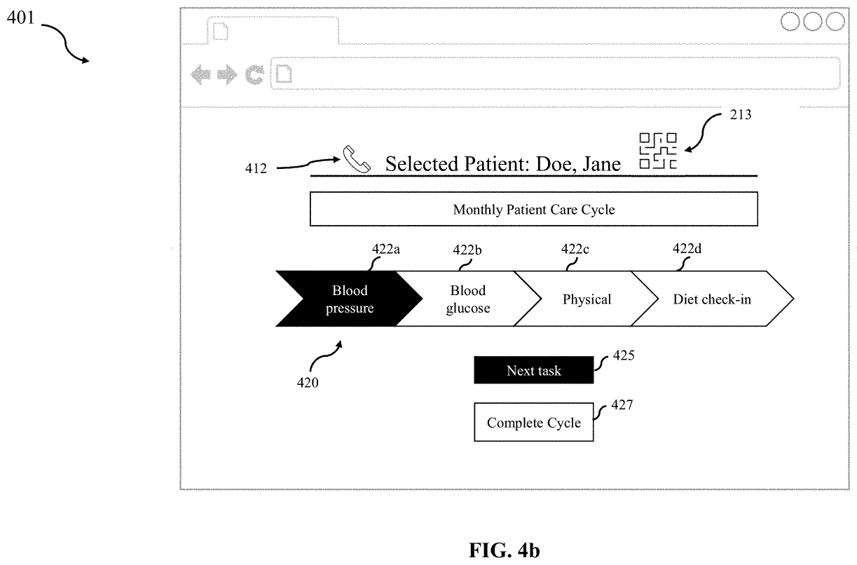

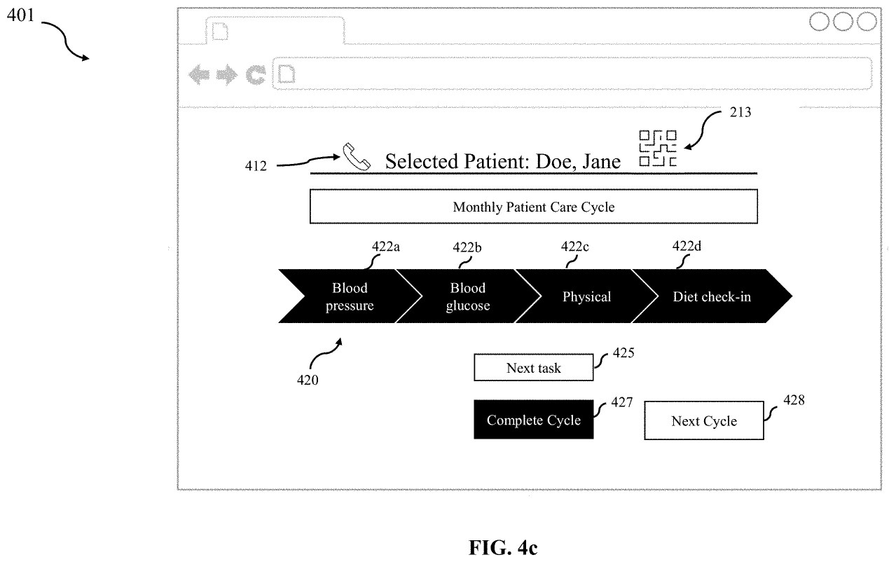

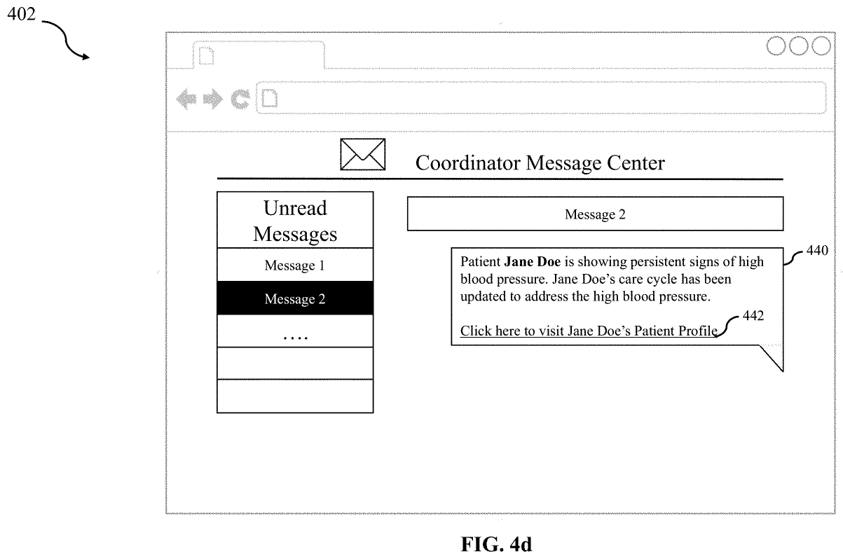

After the transmitting step 240, the particular coordinator device 105 that receives the first information 310 may then display the first custom webpage. With momentary reference to FIG. 4a, a graphical user interface of a first custom webpage 400 is shown. As illustrated, the first custom webpage includes the unique patient identifier 213. The first custom webpage 400 also comprises a plurality of regions 410 for receiving coordinator input data. The coordinator input data includes blood pressure data (systolic and diastolic), and relevant health questions such as the “time since [the patient's] last bowel movement” and “how many hours sleep [the patient got] last night.” These various medical attributes are non-limiting examples of medical attribute data that may be obtained.

Custom webpage 400 is an example of a user interface that a coordinator 104 may use. The coordinator 104 may obtain this data from the patient 110 by calling the patient directly. Calling a patient 110 may be preferred for patients that are technologically indigent in comparison to other technologic methods (e.g., obtaining this data by sending a webpage to the patient to fill out). To this end, the custom webpage 400 may include an icon 412 to initiate a phone call with the particular patient 110.

As an example, after and/or during a call with the patient 110, the coordinator 104 may simply enter in data from the patient 110 and submit the coordinator input data 258. Regardless of the method for obtaining the coordinator input data (e.g., by phone call, SMS message, by other care provider 114, etc.), the method 200 comprises receiving from the particular coordinator computing device 105, the coordinator input data 258. Generally, the coordinator input data comprises at least the (i) the unique patient identifier 213 associated with the first custom webpage, (ii) the unique coordinator identifier 223 associated with the particular coordinator computing device 105 and (iii) at least an amount of time 256 the particular coordinator computing device 105 interacted with each of the plurality of regions 410 of the first custom webpage. The coordinator input data 258 may also comprise a plurality of medical attribute data. Subsequently, the method 200 comprises receiving 250 the coordinator input data followed by storing 260, in the particular coordinator record, the coordinator input data 258.

The amount of time 256 that a particular coordinator computing device 105 interacts with each of the plurality of regions 410 of the first custom webpage may be measured in any suitable way. For instance, the amount of time 256 may be measured by an application state manager programmed into the webpage. The application state manager may detect if certain actions (e.g., clicking, typing, swiping, etc.) are being performed on the webpage. After the first detection, a timer may start. Until the coordinator has left the webpage, the timer may increase so long as the coordinator is active on the webpage as detected by the actions made on the page. However, after a certain period of time of inaction, the timer may stop, and the amount of time may be sent to server 102 to be stored in a coordinator record in attached database 103. Multiple timed periods of activity and inactivity may be tracked and monitored using the described systems and methods. Furthermore, software may be installed on the coordinator's computing device 105 that is programmed to track the coordinator's activity. Such alternatives to web-based methods are within the spirit and scope of the invention.

An additional aspect of the invention is for coordinators to operate on a cycle of care that is predetermined according to the patient's ailment(s) and needs. In this way, a coordinator may complete a first task, followed by subsequent tasks that cannot be completed unless the first task is completed. The frequency of the tasks may be weekly, monthly, yearly, etc. For instance, and with reference now to FIGS. 4b-4c, a custom webpage 401 is shown. The custom webpage 401 comprises a medical care plan 420 (depicted as a series of chevron symbols, each having a particular care activity). The medical care plan 420 comprises a plurality of activities 422a-422d. The illustrated plurality of activities 422a-422d are related to, for instance, a diabetic or pre-diabetic patient. The first activity 422a is for the coordinator to obtain blood pressure data (refer to FIG. 4a). The second activity 422b is for the coordinator to obtain blood glucose data. The blood glucose data may be, for instance, obtained directly from the patient 110 or from the patient's other care provider 114. The third activity 422c is for the coordinator to set-up a physical for the patient 110; a physical is an examination performed by a physician to observe the general health of a patient. The fourth and final activity 422d in the cycle is for the coordinator to perform a diet check-in. The diet check-in may include, for instance, having the coordinator call the patient to gather information about their nutritional habits. For a diabetic or pre-diabetic patient, the coordinator may provide feedback to the patient about their nutritional habits. For instance, if a patient is eating high carbohydrate foods that include refined sugars and/or refined carbohydrates, the coordinator may provide feedback to the patient on how to cut down on these foods that will most likely exacerbate the patient's diabetic condition.

The medical care plan 420 illustrated in FIGS. 4b-4c are examples of a cycle that a coordinator may be required to perform. In one aspect, the coordinator is unable to proceed to the next cycle until the present cycle is complete. For instance, as shown in FIG. 4a, the next task button 425 is enabled and the complete cycle button 427 is disabled. Furthermore, only the first activity 422a is complete. As shown in FIG. 4b, all of the activities 422a-422d are complete. Accordingly, the next task button 425 is disabled and the complete cycle button 427 is enabled. FIG. 4c also illustrates a next cycle button 428 that may not be engaged by user to move to the next cycle until the current cycle is completed. It is understood that these plurality of cycles may be used for the medical care plan. This graphical user interface is but one example of an interface that may be used. In sum, the systems and methods may include a medical care plan, the medical care plan may operate on a cycle that forces the coordinator to complete the cycle before moving onto a subsequent cycle. A plurality of cycles may be used, for instance, a coordinator may complete a weekly cycle, a monthly cycle, and cycles on more extended periods of time (e.g., yearly, bi-yearly, etc.). In this way the patient receives care and thereby reduces risk of hospital readmission by the patient.

In one embodiment, the method further comprises (not illustrated), prior to the transmitting 240, retrieving, with the first computing device 102, the patient record for the particular patient, generating, with the first computing device 102, third information comprising a medical care plan corresponding with the at least one medical attribute for the particular patient. In one embodiment, the medical care plan comprises a plurality of cycles. In one embodiment, each cycle of the plurality of cycles includes a plurality of activities for the coordinator device to complete. In one embodiment, each activity is completed upon receiving adequate coordinator input data into at least one of the plurality of regions of the first custom webpage, and a second cycle cannot be started until all activities of a first cycle are completed.

Adequate coordinator input data may be data that is consistent with the medical attribute being measured. For instance, adequate blood pressure data may be blood pressure data that falls between common values for blood pressure. As an example, if a coordinator inputs 1200 mmHg as the systolic blood pressure of a patient, it is unlikely that the coordinator correctly entered this data. Rather, it is likely that the coordinator accidentally reported 1200 mmHg instead of the more reasonable 120 mmHg. Adequate coordinator input data may also mean data that is complete. For instance, the coordinator may input the systolic blood pressure data in FIG. 4a without entering the diastolic blood pressure. Such data would be incomplete and inadequate for the purposes of healthcare. The task for the coordinator may be repeated to obtain this data before moving to the next activity and/or the next cycle.

After generating the third information, the method comprises transmitting, to the particular coordinator computing device, the third information. Generally, the third information is displayed on the first custom webpage proximate to the plurality of regions for receiving the coordinator input data acting as a guide for instructing the coordinator. As an example, FIGS. 4b-4c show a medical care plan for a particular patient. The illustrated embodiment shows the displayed third information, i.e., the medical care plan 420.

With reference back to the illustrated embodiments, FIG. 2c illustrates that method 200 comprises generating 270 second information 320. The second information 320 is generally associated with the particular coordinator computing device 105. The second information 320 may comprise (i) the unique coordinator identifier 223 of the particular coordinator 104, and (ii) second custom webpage information 272. The second custom webpage information 272 generally includes information for displaying a second custom webpage on a supervisor computing device 106. This information may include data such as, but not limited to, the coordinator input data 258 associated with the particular coordinator 104.

After generating the second information 320 in step 270, the method comprises transmitting 280 the second information 320 to the supervisor's computing device 106. In one embodiment, the second custom webpage received by the supervisor's computing device comprises a graphical representation of a timeline indicating, based on the coordinator input data 258 received from the particular coordinator computing device 105, (i) when the particular coordinator computing device 106 was engaging with regions on the first custom webpage over a first period of time and (ii) when the particular coordinator computing device 106 was not engaging with the regions on the first custom webpage over the first period of time. In simpler terms, the custom webpage displayed on the supervisor's computing device 106 displays information that informs the supervisor of his/her coordinator's productivity.

With reference momentarily to FIG. 5a, an example of a second custom webpage 500 is shown. The second custom webpage is displayed on a supervisor computing device 106, wherein the second custom webpage information comprises the coordinator input data associated with the particular coordinator. As illustrated, the second custom webpage 500 comprises a graphical representation of a timeline 510. The timeline includes regions that show when the coordinator is (1) active, (2) not active, and (3) taking a break. With regards to the activity of the coordinator, the first custom web page received by the coordinator 104 and displayed on the coordinator's device 105 may track the coordinator's activity. Furthermore, additional software may be used (e.g., software installed on the coordinator's device) to track activity. With respect to tracking activity on the webpage (i.e., the first custom webpage) sent to the coordinator, the activity may be tracked in relation to whether or not the coordinator was engaging in regions on the first custom webpage. Additional features may be included, such as whether or not the coordinator 104 was actively engaged in a phone call with a patient 110, or the patient's other care provider 114. The illustration of FIG. 5a shows that the coordinator's workflow is broken up into three simple categories. However, more complex categories may be used and are within the spirit and scope of the invention. For instance, the coordinator's activity may be split up into several classifications, such as activity where the coordinator was engaged with regions on the first custom webpage and activity where the coordinator was on a phone call with a patient.

Further, the methods and systems described herein may also provide unique interfaces for supervisors and/or health care providers to oversee the productivity of their respective coordinators. For instance, FIG. 5b shows a fourth custom webpage 501 that comprises a graphical representation of a plurality of parallel timelines 520. As illustrated, the graphical representation includes a first timeline 522 and a second timeline 524. The timelines are arranged in a parallel manner to each other. However, other configurations may be used and are within the spirit and scope of the invention. Nonetheless, the parallel timelines 520 allow for the supervisor (or health care provider, as the case may be) to identify gaps in availability of a group of coordinators. As noted above, it is critical that a coordinator is available to provide care in accordance with regional and federal laws and/or guidelines. Thus, a supervisor may determine from the parallel timelines in FIG. 5b that coordinators are generally unavailable from 12:00 PM to about 3:00 PM. Accordingly, the supervisor may hire an additional coordinator that can provide care during this time period to ensure that patients have appropriate access to healthcare in accordance with certain policies, regulations and guidelines.

In one embodiment, a method comprises (not illustrated), after receiving the coordinator input data from at least two coordinator computing devices, generating, with the first computing device, fourth custom webpage information for generating a fourth custom webpage 501, wherein the fourth custom webpage comprises a second graphical representation of a plurality of parallel second timelines 520, wherein each second timeline (522, 524) indicating, based on the coordinator input data received from the at least two coordinator computing devices, (i) when the particular coordinator computing device was engaging with the regions on the first custom webpage over a second period of time and (ii) when the particular coordinator computing device was not engaging with the regions on the first custom webpage over the second period of time. After the generating, the method comprises transmitting, to the supervisor computing device, said fourth custom webpage information such that the fourth custom webpage is displayed on the supervisor computing device.

As another example of a unique interface, FIG. 5c shows a custom webpage 503 that may be received by a health care provider 109 and viewed on a provider computing device 108. The custom webpage 503 comprises a graphical representation 540. The graphical representation 540 generally comprises at least one medical attribute 542. The medical attribute 542 in the illustrated embodiment is blood pressure (systolic). The medical attribute 542 data show data for at least one patient (three illustrated) taken at intervals over an extended period of time, i.e., the data is shown as a function of time. The illustrated data points are examples of coordinator input data that is received by the server 102, stored in database 103, and stored in the corresponding record associated with the patient. The data is easily accessed by the health care provider 109 and reviewed to determine what action(s) should be taken. For instance, the graphical representation 540 includes two dotted lines. The region between the dotted lines 560, 561 shows a normal range of systolic blood pressure. The normal range of systolic blood pressure is a range that would not normally require action on behalf of the health care provider 109. The region above the top dotted line 561 is a second region capable of detecting an occurrence of a trigger event when the healthcare provider engages in the region. Additionally, the region below the bottom dotted line 560 may also be a second region capable of detecting an occurrence. It is also understood that in other embodiments, second regions may also be areas proximate to and surrounding certain data points. This graphical representation provides a very simple way for the provider to view and identify when action should be taken to reduce the risk of the patient of hospital readmission.

The illustrated embodiment shows, on the final data point for patient 2, a pop-up 550 (e.g., appears after hovering over the final data point with cursor 544) that provides additional data for the final data point. As shown, the pop-up 550 includes the precise blood pressure measurement (142 mmHg), the date that the blood pressure was measured (Feb. 2, 2021), and which coordinator (Coordinator B) was responsible for recording the data point. The health care provider can simply click or engage the link, “[r]equires additional care” to initiate providing the patient with additional care. However, it is understood that other means for engaging and providing action directly from the custom webpage may be used and are within the spirit and scope of the present invention. For instance, clicking the link may initiate a cascade of messages sent to one or more of (1) the patient, (2) the other care provider, (3) the coordinator(s), and (4) supervisors (see FIG. 2b). The messages may notify the respective parties that the healthcare provider has identified a potential risk in the patient. In this particular embodiment, one such risk is that the patient is at a higher risk for heart-related complications such as a heart attack. Accordingly, the patient's blood pressure should be addressed in a manner that reduces this risk. The action message may be sent via a phone call, a SMS message, or a message sent via HTTP or HTTPS protocol as well other means for sending messages. The action item may provide the coordinator with a list of additional activates to be completed or displayed on the first custom webpage (as illustrated in FIG. 4a).

In sum, the graphical representation shown in FIG. 5c provides a simple streamlined way for identifying risk and reducing incidence of hospitalization and patient readmission by making it easy for a provider to control a patient population. Furthermore, the illustrated webpage 503 embodiment demonstrates a unique interface that a health care provider 109 may use to initiate protocols that lower risk to the patient for further complications. Moreover, the unique interface provides a method for health care providers 109 to quickly analyze important health data without a significant investment of time from the health care provider 109. Rather, the healthcare provider's coordinators and supervisors take on the responsibility of overseeing the patient's health functions (e.g., blood pressure) and the healthcare provider takes on the responsibility of choosing how to reduce the risk to the patient. Overall, the described methods are capable of improving efficiency in healthcare while reducing risk to patients and at a lower cost than is currently possible.

FIG. 4d is an example embodiment of an action message 440 that is provided to a coordinator device. As illustrated, the action message is displayed on a custom webpage 402 in a web browser on a coordinator device 105. The illustrated action message 440 informs the coordinator that a trigger event (persistent high blood pressure) has been detected and flagged by a health care provider 109. While not required in all instances of trigger events, the illustrated trigger event (persistent high blood pressure) resulted in an updated care cycle. Lastly, the coordinator 104 can readily access the patient's profile by clicking on link 442. Link 442 may redirect the coordinator to a custom webpage such as any of FIGS. 4a-4c. The action message 440 may include other, non-illustrated information, such as a graphical representation of the medical attribute in question (see, e.g., graphical representation 540 FIG. 5c).

FIG. 5d is an example embodiment of an action message that is provided to a supervisor device. As illustrated, the action message 565 is displayed on a custom webpage 504. The illustrated action message similar the supervisor of the patient's trigger event just like the custom webpage shown in FIG. 4d. In addition to the information sent to the coordinator, the custom webpage 504 sent to the supervisor informs the supervisor of which coordinator has been managing the patient's care (Coordinator A). The action message 565 also includes links 566, 567 that direct the supervisor to the profile of the coordinator and patient, respectively. The custom webpage 504 may additionally include a timeline 568 of the coordinator responsible for the patient's care in the action message 565. In this way, the supervisor can readily determine if that particular coordinator is being overworked (i.e., does not have enough time to effectively provide medical care to the patient) or if the coordinator is available to care for the patent. The custom webpage 504 may also additionally include a medical care plan 569 for the particular patient in the action message 565. In this way, the supervisor can determine what portion of a medical care plan the patient has completed (through the coordinator) and if the medical care plan should be adjusted to improve the patient's ailment. Additional graphical representations (not illustrated) may be used, such as a graphical representation of a medical attribute (see, e.g., graphical representation 540 of FIG. 5c).

Lastly, FIG. 5e is an example embodiment of an action message that is provided to a patient computing device 111. The illustrated embodiment shows a mobile device 505 and an action message 575 received by the mobile device 505 as an SMS message. The action message 575 asks the patient if they have taken their blood pressure medication today. The patient can respond with a YES or a NO to indicate their response, although the server may be programmed to accept other answers, such as “I don't know” and alternative phrasings to the words “yes” and “no” (e.g., “yeah” and “nope”). After receiving the message from the patient, the server may send a message to the coordinator(s), supervisor(s), and/or health care providers, such as the action message depicted in FIGS. 4d and 5d. It is noted that action message 575 may similarly be displayed on a wearable device 112, such as an activity band.

As previously described, the fourth custom webpage may comprise a third graphical representation 540, the third graphical representation comprising at least one medical attribute 542 of the particular patient taken at intervals over an extended period of time. Furthermore, the third graphical representation 540 may comprise a plurality of displayed second regions, wherein each of the plurality of displayed second regions is capable of detecting an occurrence of a trigger event, wherein the trigger event only occurs when the provider computing device engages with at least one particular second region on the fourth custom webpage. For example, in one embodiment a trigger event may only occur when provider engages with data points with the region outside dotted lines 560, 561 which is indicative of an abnormal measurement. For example, a trigger event may be provided by the provider selecting an icon or other graphical representation on the display by interfacing with a screen, such as by using a swipe or swiping gesture, a push or other means of indicating a trigger event.

With momentary reference to FIG. 3b in particular, in one embodiment, a method comprises generating, with the first computing device, fourth information 330 comprising (i) the patient identifier for the particular patient, (ii) and fourth custom webpage information for providing a fourth custom webpage to a provider computing device, b) transmitting, to a provider computing device of a provider, the fourth information for displaying the fourth custom webpage, c) receiving, from the provider computing device, provider input data 340, wherein the provider input data is received after the occurrence of the trigger event is detected and wherein the provider input data comprises (i) a unique provider identifier of the provider associated with the trigger event; (ii) the unique patient identifier of the particular patient; (iii) second region information associated with where the trigger event occurred, d) generating, with the first computing device, fifth information 350 comprising (i) the unique patient identifier, (ii) the unique provider identifier of the provider computing device associated with the trigger event; (iii) the second region information; (iv) an action message comprising written instructions for the particular patient, and e) transmitting, to at least one of (i) a patient computing device associated with the particular patient, (ii) the particular coordinator computing device, and (iii) the supervisor computing device, the fifth information 350.

In one embodiment, the trigger event is that the patient did not complete a HbA1C test for more than three months. In one embodiment, the trigger event is that the patient is complaining of the side effect of a current medication. In one embodiment, the trigger event is that the patient is compliant with diabetic medication that is prescribed but still has high blood glucose levels. In one embodiment, the trigger event is that the patient is not compliant with the diabetic medication but has a stable blood glucose level. In one embodiment, the trigger event is that the patient is diabetic and is not measuring blood glucose regularly. In one embodiment, the trigger event is that the patient is diabetic and has no blood glucose monitoring kit. In one embodiment, the trigger event is that the patient is diabetic, and the patient's last ophthalmologist visit was more than one year ago. In one embodiment, the trigger event is that the patient has diabetic neuropathy symptoms but is not seeing a neurologist and/or a podiatrist. In one embodiment, the trigger event is that the patient has diabetic neuropathy symptoms, but the patient's last visit was more than three months ago. In one embodiment, the trigger event is that the patient is diabetic and the last time the patient was screened for nephropathy was in the last 6 months.

In one embodiment, the trigger event is that the patient is diabetic, but the patient's last appointment with a podiatrist was more than six months ago. In one embodiment, the trigger event is that the patient is diabetic and having symptoms of (symptomatic, neuropathic, dermopathic) foot, but is not seeing a neurologist and/or a podiatrist. In one embodiment, the trigger event is that the patient is diabetic and having symptoms of deformed foot but is not seeing an orthopedic. In one embodiment, the trigger event is that the patient is diabetic and having symptoms of (ulcerated, distressed, arthropathic, gangrenous, and/or terminal) foot, but is NOT seeing a vascular specialist and/or a vascular surgeon. In one embodiment, the trigger event is that the patient is diabetic and has uncontrolled blood sugar but has not seen a podiatrist within the last three months.

In one embodiment, the trigger event is that the patient is compliant with the anti-hypertensive medication that was prescribed but has 3 or more consecutive measurements of blood pressure 160/90 mmHg or higher. In one embodiment, the trigger event is that the patient is NOT compliant with the anti-hypertensive medication(s) prescribed, but the blood pressure readings are within a normal range. In one embodiment, the trigger event is that the patient is hypertensive and is not measuring their blood pressure regularly. In one embodiment, the trigger event is that the patient is hypertensive and has no blood pressure monitoring kit. In one embodiment, the trigger event is that the patient is hypertensive and has not visited a cardiologist in the last year.

In one embodiment, the trigger event is that the patient is compliant with the sleeping medication that has been prescribed, but still has a sleeping problem. In one embodiment, the trigger event is that the patient complaining of sleeping problems and has no sleeping medication.

In one embodiment, the trigger event is that the patient's lipid profile test is not within a normal range and the patient takes statins. In one embodiment, the trigger event is that the patient's lipid profile test is not within a normal range and the patient is not taking any statins. In one embodiment, the trigger event is that the patient has hyperlipidemia and the last time he did lipid profile test was more than six months ago. In one embodiment, the trigger event is that the patient has hyperlipidemia and the last visit to a cardiologist was more than one year ago. In one embodiment, the trigger event is that the patient has hyperlipidemia as well as an abnormal lipid panel, but no statins have been prescribed to the patient. In one embodiment, the trigger event is that the patient has an abnormal lipid panel, but no statins have been prescribed to the patient. In one embodiment, the trigger event is that the patient has some out of a normal range results, or worse results for the lipid panel, and is compliant with lipid lowering medications that have been prescribed.

In one embodiment, the trigger event is that the patient having COVID-19 symptoms and did not do a COVID-19 RT-PCR test. In one embodiment, the trigger event is that the patient has COVID-19 related symptoms but was not tested for COVID-19. In one embodiment, the trigger event is that the patient has a confirmed result of a COVID-19 positive test, the patient is experiencing fever, but has taken any antipyretic medications. In one embodiment, the trigger event is that the patient has a confirmed result of a COVID-19 positive test, the patient is experiencing cough, but not taking any antitussives and/or expectorants. In one embodiment, the trigger event is that the patient has a confirmed result of a COVID-19 positive test, the patient is experiencing dyspnea, but is not taking any bronchodilators. In one embodiment, the trigger event is that the patient has a confirmed result of a COVID-19 positive test, the patient is experiencing a sore throat, but not taking any pain relief medications. In one embodiment, the trigger event is that the patient has a confirmed result of a COVID-19 positive test, the patient is experiencing worsening of symptoms and/or aggravated exacerbations. In one embodiment, the trigger event is that the patient did not follow his/her scheduled immunization/vaccination. In one embodiment, the trigger event is that the patient complains of a shortness of breath. In one embodiment, the trigger event is that the patient has memory loss. In one embodiment, the trigger event is that the patient complains of chest pain.

In one embodiment, the trigger event is that the patient did not visit the doctor for more than three months. In one embodiment, the trigger event is that the patient did not visit the doctor for more than six months. In one embodiment, the trigger event is that the patient did not visit the doctor for more than one year. In one embodiment, the trigger event is that the patient is not satisfied with his/her medical condition and did not visit a doctor.

In one embodiment, the trigger event is that the patient suffering from obesity (e.g., a body mass index of greater than 25 kg/m²). In one embodiment, the trigger event is that the patient had his last body mass index (“BMI”) check, more than three months ago. In one embodiment, the trigger event is that the patient has a BMI of less than 18.5 kg/m² but does not have any specific diet plan. In one embodiment, the trigger event is that the patient has a BMI between 25 and 30 kg/m² but does not have any specific diet. In one embodiment, the trigger event is that the patient has a BMI between 25 and 30 kg/m², and has limitations performing physical activities. In one embodiment, the trigger event is that the patient has a BMI between 25 and 30 kg/m², and has limitations performing physical activities, and needs durable medical equipment (“DME”) supply or support. In one embodiment, the trigger event is that the patient has a BMI of more than 30 kg/m² but does not have any specific diet. In one embodiment, the trigger event is that the patient has a BMI of more than 30 kg/m², and has limitations performing physical activities. In one embodiment, the trigger event is that the patient has a BMI of more than 30 kg/m², and has limitations performing physical activities, and needs DME supply or support. In one embodiment, the trigger event is that the patient has a BMI of more than 30 kg/m² and has not had a lipid profile screening in more than a year. In one embodiment, the trigger event is that the patient has a BMI of more than 30 kg/m², has an abnormal lipid profile test, and has not seen a cardiologist.

In one embodiment, the trigger event is that the patient has chronic kidney disease and the patient's last labs for kidney function was more than three months ago. In one embodiment, the trigger event is that the patient has liver disease and has not had a recent (e.g., greater than a month) liver function test. In one embodiment, the trigger event is that the patient complains of cyanosis discoloration (e.g., bruise-like discolorations). In one embodiment, the trigger event is that the patient has not visited an endocrinologist within the last year. In one embodiment, the trigger event is that the patient has controlled blood glucose and shows no signs of kidney damage, but the patient's last kidney function test (nephrology screening) was done more than six months ago. In one embodiment, the trigger event is that the patient has controlled blood glucose and shows signs or symptoms of kidney damage, but the patient's last kidney function test (nephrology screening) was done more than three months ago. In one embodiment, the trigger event is that the patient has uncontrolled blood glucose and shows no signs of kidney damage, but the patient's last kidney function test (nephrology screening) was done more than three months ago. In one embodiment, the trigger event is that the patient has uncontrolled blood glucose and shows signs or symptoms of kidney damage, but the patient's last kidney function test (nephrology screening) was done more than three months ago. In one embodiment, the trigger event is that the patient has estimated glomerular filtration rate (“eGFR”) of less than 30 but is still taking metformin. In one embodiment, the trigger event is that the patient has an eGFR<30 but is not seeing a nephrologist. In one embodiment, the trigger event is that the patient has an eGFR of 30-44 but is not seeing a nephrologist. In one embodiment, the trigger event is that the patient has an albumin-to-creatinine ratio (“ACR”) of 300 or more but is not seeing a nephrologist. In one embodiment, the trigger event is that the patient has an eGFR of 45-59 but is not seeing a nephrologist. In one embodiment, the trigger event is that the patient has an ACR between 30 and 300 but is not seeing a nephrologist. In one embodiment, the trigger event is that the patient has an eGFR<59 but does not have a specific diet plan for diabetes. In one embodiment, the trigger event is that the patient has ACR of 30 or more but does not have a specific diet plan for diabetes. In one embodiment, the trigger event is that the patient has eGFR<59 but is not seeing a cardiologist. In one embodiment, the trigger event is that the patient has ACR of 30 or more but is not seeing a cardiologist.

In one embodiment, the trigger event is that the patient had an accident and has difficulty in walking. In one embodiment, the trigger event is that the patient can't do his activities of daily living (“ADL”) and instrumental activities of daily living (“IADL”) independently and does not have a home health aid. In one embodiment, the trigger event is that the patient has osteoporosis and the patient's last bone-density test was more than one year ago. In one embodiment, the trigger event is that the patient has severe pain, despite being compliant with the patient's prescribed medication regimen. In one embodiment, the trigger event is that the patient having pain, but is out of prescribed painkillers. In one embodiment, the trigger event is that the patient is on blood thinners and is experiencing bleedings or bruising. In one embodiment, the trigger event is that the patient's vision is worsening. In one embodiment, the trigger event is that the patient has speech difficulties. In one embodiment, the trigger event is that the patient reports symptoms of skin disorders (rash, lesions, itching, etc.). In one embodiment, the trigger event is that the patient complains of urinary and/or fecal incontinence. In one embodiment, the trigger event is that the patient experienced two or more incidents of falls and does not have enough home assistance to prevent any future falls. In one embodiment, the trigger event is that the patient has experienced at least one incident of fall where physical injury was reported, and the patient does not have enough home assistance to prevent future falls. In one embodiment, the trigger event is that the patient experienced an incident of one or more of severe dizziness, lightheadedness, and drowsiness. In one embodiment, the trigger event is that the patient is requesting more hours of home health aid.

In one embodiment, the trigger event is that the patient did not undergo colonoscopy/mammography according to an approved protocol. In one embodiment, the trigger event is that the patient complains of numbness. In one embodiment, the trigger event is that the patient does not know the dosage or duration of one or more prescribed medications. In one embodiment, the trigger event is that the patient complains of heartburn. In one embodiment, the trigger event is that the patient complains of abdominal pain. In one embodiment, the trigger event is that the patient complains of constipation or diarrhea. In one embodiment, the trigger event is that the patient complains of seizures. In one embodiment, the trigger event is that the patient complains of muscle cramps. In one embodiment, the trigger event is that the patient is complaining about malodorous or discoloration of urine, or painful urination. In one embodiment, the trigger event is that the patient is complaining of swelling about one or more of extremities.

In one embodiment, the trigger event is that the patient has a pacemaker or implantable cardiovert defibrillator (“ICD”) and has not visited the cardiologist for more than six months.

In one embodiment, the trigger event is that the patient has depression symptoms but does not have a psychiatrist. In one embodiment, the trigger event is that the patient has depression symptoms, but his last visit was more than three months ago. In one embodiment, the trigger event is that the patient has depression symptoms but does not have enough home health aid for support. In one embodiment, the trigger event is that the patient is having worsening depression symptoms although the patient is compliant with anti-depressive prescribed medications. In one embodiment, the trigger event is that the patient has depression symptoms but does not have any anti-depressive medications prescribed. In one embodiment, the trigger event is that the patients last patient health questionare-9 (“PHQ-9”) depression test was done more than one year ago.

In one embodiment, the trigger event is that the patient is experiencing deterioration in vision. In one embodiment, the trigger event is that the patient complains of hearing impairment but does not have one or more of an ear, nose, throat (“ENT”) specialist, an audiologist and a hearing aid dispenser.

### iii. Systems

In one embodiment, a system for decreasing incidence of an uncontrolled medical attribute of a plurality of patients in a patient population comprises: (a) a first server comprising at least one processor, (b) a particular coordinator computing device associated with a particular coordinator, and (c) a supervisor computing device associated with a particular supervisor. In one embodiment, the system comprises a provider computing device. Additional computing devices, such as wearable device 112, and other care provider computing device 115 may additionally be included in such systems.

In one embodiment, the at least one processor is configured for generating and storing, in an attached database, a patient record for each of the plurality of patients and a coordinator record. In one embodiment, the at least one processor is configured for generating first information associated with a particular patient from the plurality of patients. The first information may comprise (i) a unique patient identifier of the particular patient and (ii) first custom webpage information for displaying a first custom webpage comprising a plurality of regions for receiving coordinator input data from a particular coordinator computing device. In one embodiment, the at least one processor is configured for generating second information associated with the particular coordinator computing device, wherein the second information comprises (i) a unique coordinator identifier of a particular coordinator, and (ii) second custom webpage information for displaying a second custom webpage on a supervisor computing device, wherein the second custom webpage information comprises the coordinator input data associated with the particular coordinator.

In one embodiment, the at least one processor is configured for transmitting the first information to the particular coordinator computing device. In one embodiment, the at least one processor is configured for transmitting the second information to the supervisor computing device. In one embodiment, the at least one processor is configured for generating third information, wherein the third information comprises a medical care plan corresponding with the uncontrolled medical attribute for the particular patient. In one embodiment, the medical care plan comprises a plurality of cycles, wherein each cycle includes a plurality of activities for the particular coordinator computing device to complete, wherein each activity is completed upon receiving adequate coordinator input data into at least one of the plurality of regions of the first custom webpage, and wherein a second cycle cannot be started until all activities of a first cycle are completed. In one embodiment, the at least one processor is configured for transmitting, to the particular coordinator computing device, the third information. In one embodiment, the at least one processor of the first server is configured for generating fourth information comprising a patient identifier for the particular patient, and fourth custom webpage information for a fourth custom webpage. In one embodiment, the at least one processor of the first server is configured for transmitting the fourth information to the provider computing device. In one embodiment, the at least one processor of the first server is configured for receiving, from the provider computing device, provider input data.

In one embodiment, the particular coordinator computing device is configured for receiving, from the first server, the first information. In one embodiment, the particular coordinator computing device is configured for displaying the first custom webpage. In one embodiment, the particular coordinator computing device is configured for receiving, from the particular coordinator, the coordinator input data. In one embodiment, the particular coordinator computing device is configured for transmitting, to the first server the coordinator input data. In one embodiment, the coordinator input data comprises (i) the unique patient identifier associated with the first custom webpage, (ii) the unique coordinator identifier associated with the particular coordinator computing device and (iii) at least an amount of time the particular coordinator computing device interacted with each of the plurality of regions of the first custom webpage. In one embodiment, the particular coordinator computing device is configured for displaying the third information on the first custom webpage proximate to the plurality of regions for receiving the coordinator input data acting as a guide for instructing coordinator. In one embodiment, the particular coordinator computing device is configured for displaying the third information on the first custom webpage.

In one embodiment, the supervisor computing device is configured for receiving, from the first server, the second information. In one embodiment, the supervisor computing device is configured for displaying the second custom webpage.

In one embodiment, the provider computing device is configured for receiving, from the first server, the fourth information.

In one embodiment, the provider computing device is configured for displaying the fourth custom webpage.

In one embodiment, the provider computing device is configured for receiving, from the provider, the provider input data. In one embodiment, the provider input data comprises (i) a unique provider identifier of the provider associated with a trigger event, (ii) the unique patient identifier of the particular patient, and (iii) second region information associated with where the trigger event occurred.

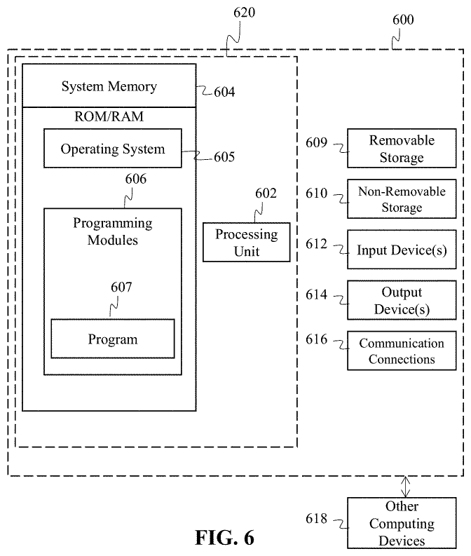

As referenced above, the methods described may be implemented on at least one processor, such as at least one processor on a server. Devices (102, 105, 106, 108, 111, 112, 115) may be included in the above-described systems and utilized in the above-described methods. The at least one processor may be included as a part of a computing device or may also be a device performing some or all of functions of a computing device. Referring now to FIG. 6, a computing device 600 is shown. FIG. 6 is a block diagram of a system including an example computing device 600 and other computing devices. Consistent with the embodiments described herein, the aforementioned actions performed by the various servers, devices, etc. may be implemented in a computing device, such as the computing device 600 of FIG. 6. Any suitable combination of hardware, software, or firmware may be used to implement the computing device 600. Furthermore, computing device 600 may comprise or be included in the operating environment (e.g., shown by system 100) and processes and dataflow as described above. However, processes described above may operate in other environments and are not limited to computing device 600. Furthermore, computing device 600 may comprise an operating environment for system 100. Processes, data related to system 100 may operate in other environments and are not limited to computing device 600.

A system consistent with the invention may include a plurality of computing devices, such as computing device 600. In a basic configuration, computing device 600 may include at least one processing unit 602 and a system memory 604. Depending on the configuration and type of computing device, system memory 604 may comprise, but is not limited to, volatile (e.g., random access memory (RAM)), non-volatile (e.g., read-only memory (ROM)), flash memory, or any combination or memory. System memory 604 may include operating system 605, and one or more programming modules 606. Operating system 605, for example, may be suitable for controlling computing device 600′s operation. In one embodiment, programming modules 606 may include, for example, a program module 606 for executing the actions of system 100. Furthermore, embodiments of the invention may be practiced in conjunction with a graphics library, other operating systems, or any other application program 607 and is not limited to any particular application or system. This basic configuration is illustrated in FIG. 6 by those components within a dashed line 620.

Computing device 600 may have additional features or functionality. For example, computing device 600 may also include additional data storage devices (removable and/or non-removable) such as, for example, magnetic disks, optical disks, or tape. Such additional storage is illustrated in FIG. 6 by a removable storage 609 and a non-removable storage 610. Computer storage media may include volatile and nonvolatile, removable, and non-removable media implemented in any method or technology for storage of information, such as computer readable instructions, data structures, program modules, or other data. System memory 604, removable storage 609, and non-removable storage 610 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information, and which can be accessed by computing device 600. Any such computer storage media may be part of system 600. Computing device 600 may also have input device(s) 612 such as a keyboard, a mouse, a pen, a sound input device, a camera, a touch input device, etc. Output device(s) 614 such as a display, speakers, a printer, etc. may also be included. The aforementioned devices are only examples, and other devices may be added or substituted.

Computing device 600 may also contain a communication connection 616 that may allow system 100 to communicate with other computing devices 618, such as over a network 101 in a distributed computing environment, for example, an intranet or the Internet. Communication connection 616 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both computer storage media and communication media.

As stated above, a number of program modules and data files may be stored in system memory 604, including operating system 605. While executing on processing unit 602, programming modules (e.g., program module 606) may perform processes including, for example, one or more of the stages of a process. The aforementioned processes are examples, and processing unit 602 may perform other processes. The aforementioned processes are examples, and processing unit 602 may perform other processes and may also be configured to provide graphical user interfaces displayed associated with devices explained above. Other programming modules that may be used in accordance with embodiments of the present invention may include electronic mail and contacts applications, activity tracking applications, word processing applications, spreadsheet applications, database applications, slide presentation applications, drawing or computer-aided application programs, etc.

Generally, consistent with embodiments of the invention, program modules may include routines, programs 607, components, data structures, and other types of structures that may perform particular tasks or that may implement particular abstract data types. Moreover, embodiments of the invention may be practiced with other computer system configurations, including hand-held devices, multiprocessor systems, microprocessor-based or programmable consumer electronics, minicomputers, mainframe computers, and the like. Embodiments of the invention may also be practiced in distributed computing environments where tasks are performed by remote processing devices that are linked through a communications network. In a distributed computing environment, program modules may be located in both local and remote memory storage devices.

Furthermore, embodiments of the invention may be practiced in an electrical circuit comprising discrete electronic elements, packaged, or integrated electronic chips containing logic gates, a circuit utilizing a microprocessor, or on a single chip (such as a System on Chip) containing electronic elements or microprocessors. Embodiments of the invention may also be practiced using other technologies capable of performing logical operations such as, for example, AND, OR, and NOT, including but not limited to mechanical, optical, fluidic, and quantum technologies. In addition, embodiments of the invention may be practiced within a general-purpose computer or in any other circuits or systems.

Embodiments of the present invention, for example, are described above with reference to block diagrams and/or operational illustrations of methods, systems, and computer program products according to embodiments of the invention. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the invention have been described, other embodiments may exist. Furthermore, although embodiments of the present invention have been described as being associated with data stored in memory and other storage mediums, data can also be stored on or read from other types of computer-readable media, such as secondary storage devices, like hard disks, floppy disks, or a CD-ROM, or other forms of RAM or ROM. Further, the disclosed methods' stages may be modified in any manner, including by reordering stages, and/or inserting or deleting stages, without departing from the invention.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

We claim:

What is claimed is:

1. A computer implemented method executed by a first computing device for decreasing incidence of an uncontrolled medical attribute of a plurality of patients in a patient population thereby identifying risk and reducing incidence of hospitalization and patient readmission, the method comprising:
a. generating a patient record for each of the plurality of patients;
b. storing, in an attached database and in the patient record for each of the plurality of patients, (i) a unique patient identifier for each patient, (ii) patient identifying information and (iii) patient medical data, wherein the patient medical data comprises measurements related to at least one medical attribute of the patient;
c. generating a coordinator record for each of a plurality of coordinator devices, wherein each coordinator device is associated with a coordinator;
d. storing, in the attached database, in the coordinator record, wherein the coordinator record comprises a unique coordinator identifier associated with the particular coordinator;
e. generating first information associated with a particular patient from the plurality of patients, wherein the first information comprises (i) the unique patient identifier of the particular patient and (ii) first custom webpage information for displaying a first custom webpage comprising a plurality of regions for receiving coordinator input data from a particular coordinator computing device;
f. transmitting, to the particular coordinator computing device, the first information to have the first custom webpage displayed on the particular coordinator computing device;
g. receiving, from the particular coordinator computing device, the coordinator input data, wherein the coordinator input data comprises (i) the unique patient identifier associated with the first custom webpage, (ii) the unique coordinator identifier associated with the particular coordinator computing device and (iii) at least an amount of time the particular coordinator computing device interacted with each of the plurality of regions of the first custom webpage;
h. storing, in the particular coordinator record, the coordinator input data;
i. generating second information associated with the particular coordinator computing device, wherein the second information comprises (i) the unique coordinator identifier of the particular coordinator; and (ii) second custom webpage information for displaying a second custom webpage on a supervisor computing device, wherein the second custom webpage information comprises the coordinator input data associated with the particular coordinator;
j. transmitting, to the supervisor computing device, the second information to be displayed on the supervisor computing device, wherein the second custom webpage comprises a first graphical representation of a timeline indicating, based on the coordinator input data received from the particular coordinator computing device, (i) when the particular coordinator computing device was engaging with regions on the first custom webpage over a first period of time and (ii) when the particular coordinator computing device was not engaging with the regions on the first custom webpage over the first period of time.

2. The method of claim 1, wherein, prior to transmitting to the particular coordinator computing device the first information for the first custom webpage, the method comprises:
a. retrieving, with the first computing device, the patient record for the particular patient;
b. generating, with the first computing device, third information comprising a medical care plan corresponding with the at least one medical attribute for the particular patient;
wherein the medical care plan comprises a plurality of cycles;
wherein each cycle includes a plurality of activities for the coordinator device to complete;
wherein each activity is completed upon receiving adequate coordinator input data into at least one of the plurality of regions of the first custom webpage; and,
wherein a second cycle cannot be started until all activities of a first cycle are completed; and,
c. transmitting, to the particular coordinator computing device, the third information, wherein the third information is displayed on the first custom webpage proximate to the plurality of regions for receiving the coordinator input data acting as a guide for instructing coordinator.

3. The method of claim 1, wherein after receiving the coordinator input data from at least two coordinator computing devices, the method comprises:
generating, with the first computing device, fourth custom webpage information for generating a fourth custom webpage, wherein the fourth custom webpage comprises a second graphical representation of a plurality of parallel second timelines, wherein each second timeline indicating, based on the coordinator input data received from the at least two coordinator computing devices, (i) when the particular coordinator computing device was engaging with the regions on the first custom webpage over a second period of time and (ii) when the particular coordinator computing device was not engaging with the regions on the first custom webpage over the second period of time;
transmitting, to the supervisor computing device, said fourth custom webpage information such that the fourth custom webpage is displayed on the supervisor computing device.

4. The method of claim 2, wherein the method further comprises:
a) generating, with the first computing device, fourth information comprising (i) the patient identifier for the particular patient, (ii) and fourth custom webpage information for providing a fourth custom webpage to a provider computing device;
wherein the fourth custom webpage comprises a third graphical representation, the third graphical representation comprising at least one medical attribute of the particular patient taken at intervals over an extended period of time; and,
wherein the third graphical representation comprises a plurality of displayed second regions, wherein each of the plurality of displayed second regions is capable of detecting an occurrence of a trigger event, wherein the trigger event only occurs when the provider computing device engages with at least one particular second region on the fourth custom webpage;
b) transmitting, to a provider computing device of a provider, the fourth information for displaying the fourth custom webpage;
c) receiving, from the provider computing device, provider input data, wherein the provider input data is received after the occurrence of the trigger event is detected and wherein the provider input data comprises (i) a unique provider identifier of the provider associated with the trigger event; (ii) the unique patient identifier of the particular patient; (iii) second region information associated with where the trigger event occurred;
d) generating, with the first computing device, fifth information comprising (i) the unique patient identifier, (ii) the unique provider identifier of the provider computing device associated with the trigger event; (iii) the second region information; (iv) an action message comprising written instructions for the particular patient; and,
e) transmitting, to at least one of (i) a patient computing device associated with the particular patient, (ii) the particular coordinator computing device, and (iii) the supervisor computing device, the fifth information.

5. The method of claim 1, wherein prior to generating the patient record for the particular patient, receiving from the patient computing device at the direction of the patient, (i) the unique patient identifier of the particular patient, (ii) the patient identifying information of the particular patient and (iii) the patient medical data of the particular patient.

6. The method of claim 1, wherein the patient identifying information comprises at least one of a patient first name, a patient last name, a patient age, a patient email address, a patient unique identifier, a patient contact number, and a patient physical address.

7. The method of claim 1, wherein the patient medical data comprises at least one of communications between the particular coordinator computing device and the provider computing device, communications between the patient computing device and the particular coordinator computing device, patient medical history, patient prescription information, patient surgical information or any medical information related to the patient.

8. The method of claim 1, wherein the at least one medical attribute comprises at least one of blood glucose level, blood pressure, and insulin level.

9. The method of claim 1, wherein the transmitting step (f) comprises transmitting via at least one of HTTP and HTTPS.

10. A computer implemented method executed by a first computing device for decreasing incidence of an uncontrolled medical attribute of a plurality of patients in a patient population thereby identifying risk and reducing incidence of hospitalization and patient readmission, the method comprising:
a. generating a patient record for each of the plurality of patients;
b. storing, in an attached database and in the patient record for each of the plurality of patients, (i) a unique patient identifier for each patient, (ii) patient identifying information and (iii) patient medical data, wherein the patient medical data comprises measurements related to at least one medical attribute of the patient;
c. generating a coordinator record for each of a plurality of coordinator devices, wherein each coordinator device is associated with a coordinator;
d. storing, in the attached database, the coordinator record, wherein the coordinator record comprises a unique coordinator identifier associated with a particular coordinator;
e. generating first information associated with a particular patient from the plurality of patients, wherein the first information comprises (i) the unique patient identifier of the particular patient and (ii) first custom webpage information for displaying a first custom webpage comprising a plurality of regions for receiving coordinator input data from a particular coordinator computing device;
f. retrieving, with the first computing device, the patient record for the particular patient;
g. generating, with the first computing device, third information comprising a medical care plan corresponding with the at least one medical attribute for the particular patient;
wherein the medical care plan comprises a plurality of cycles;
wherein each cycle includes a plurality of activities for the coordinator device to complete;
wherein each activity is completed upon receiving adequate coordinator input data into at least one of the plurality of regions of the first custom webpage; and,
wherein a second cycle cannot be started until all activities of a first cycle are completed; and,
h. transmitting, to the particular coordinator computing device, the third information, wherein the third information is displayed on the first custom webpage proximate to the plurality of regions for receiving the coordinator input data acting as a guide for instructing the coordinator;
i. transmitting, to the particular coordinator computing device, the first information to have the first custom webpage displayed on the particular coordinator computing device;
j. receiving, from the particular coordinator computing device, the coordinator input data, where the coordinator input data comprises (i) the unique patient identifier associated with the first custom webpage, (ii) the unique coordinator identifier associated with the particular coordinator computing device and (iii) at least an amount of time the particular coordinator computing device interacted with each of the plurality of regions of the first custom webpage;
k. storing, in a particular coordinator record, the coordinator input data;
l. generating second information associated with the particular coordinator computing device, wherein the second information comprises (i) the unique coordinator identifier of the particular coordinator; and (ii) second custom webpage information for displaying a second custom webpage on a supervisor computing device, wherein the second custom webpage information comprises the coordinator input data associated with the particular coordinator;
m. transmitting, to the supervisor computing device, the second information to be displayed on the supervisor computing device, wherein the second custom webpage comprises a first graphical representation of a timeline indicating, based on the coordinator input data received from the particular coordinator computing device, (i) when the particular coordinator computing device was engaging with regions on the first custom webpage over a first period of time and (ii) when the particular coordinator computing device was not engaging with the regions on the first custom webpage over the first period of time.

11. The method of claim 10, wherein after receiving the coordinator input data from at least two coordinator computing devices, the method comprises:
generating, with the first computing device, fourth custom webpage information for generating a fourth custom webpage, wherein the fourth custom webpage comprises a second graphical representation of a plurality of parallel second timelines, wherein each second timeline indicating, based on the coordinator input data received from the at least two coordinator computing devices, (i) when the particular coordinator computing device was engaging with the regions on the first custom webpage over a second period of time and (ii) when the particular coordinator computing device was not engaging with the regions on the first custom webpage over the second period of time;
transmitting, to the supervisor computing device, said fourth custom webpage information such that the fourth custom webpage is displayed on the supervisor computing device.

12. The method of claim 10, wherein the method further comprises:
a) generating, with the first computing device, fourth information comprising (i) a patient identifier for the particular patient, (ii) and fourth custom webpage information for providing a fourth custom webpage to a provider computing device;
wherein the fourth custom webpage comprises a third graphical representation, the third graphical representation comprising the at least one medical attribute of the particular patient taken at intervals over an extended period of time; and,
wherein the third graphical representation comprises a plurality of displayed second regions, wherein each of the plurality of displayed second regions is capable of detecting an occurrence of a trigger event, wherein the trigger event only occurs when the provider computing device engages with at least one particular second region on the fourth custom webpage;
b) transmitting, to the provider computing device of a provider, the fourth information for displaying the fourth custom webpage;
c) receiving, from the provider computing device, provider input data, wherein the provider input data is received after the occurrence of the trigger event is detected and wherein the provider input data comprises (i) a unique provider identifier of the provider associated with the trigger event; (ii) the unique patient identifier of the particular patient; (iii) second region information associated with where the trigger event occurred;
d) generating, with the first computing device, fifth information comprising (i) the unique patient identifier, (ii) the unique provider identifier of the provider computing device associated with the trigger event; (iii) the second region information; (iv) an action message comprising written instructions for the particular patient; and,
e) transmitting, to at least one of (i) a patient computing device associated with the particular patient, (ii) the particular coordinator computing device, and (iii) the supervisor computing device, the fifth information.

13. The method of claim 10, wherein prior to generating the patient record for the particular patient, receiving from a patient computing device at the direction of the patient, (i) the unique patient identifier of the particular patient, (ii) the patient identifying information of the particular patient and (iii) the patient medical data of the particular patient.

14. The method of claim 10, wherein the patient identifying information comprises at least one of a patient first name, a patient last name, a patient age, a patient email address, a patient unique identifier, a patient contact number, and a patient physical address.

15. The method of claim 10, wherein the patient medical data comprises at least one of communications between the particular coordinator computing device and a provider computing device, communications between a patient computing device and the particular coordinator computing device, patient medical history, patient prescription information, patient surgical information or any medical information related to the patient.

16. The method of claim 10, wherein the at least one medical attribute comprises at least one of blood glucose level, blood pressure, and insulin level.

17. A system for decreasing incidence of an uncontrolled medical attribute of a plurality of patients in a patient population, the system comprising:
(a) a first server comprising at least one processor, wherein the at least one processor is configured for:
(i) generating and storing, in an attached database, a patient record for each of the plurality of patients and a coordinator record;
(ii) generating first information associated with a particular patient from the plurality of patients, wherein the first information comprises (A) a unique patient identifier of the particular patient and (B) first custom webpage information for displaying a first custom webpage comprising a plurality of regions for receiving coordinator input data from a particular coordinator computing device;
(iii) generating second information associated with the particular coordinator computing device, wherein the second information comprises (A) a unique coordinator identifier of a particular coordinator; and (B) second custom webpage information for displaying a second custom webpage on a supervisor computing device, wherein the second custom webpage information comprises the coordinator input data associated with the particular coordinator;
(iv) transmitting the first information to the particular coordinator computing device, and the second information to the supervisor computing device;
(b) the particular coordinator computing device, wherein the particular coordinator computing device is associated with the particular coordinator, and wherein the particular coordinator computing device is configured for:
(i) receiving, from the first server, the first information;
(ii) displaying the first custom webpage;
(iii) receiving, from the particular coordinator, the coordinator input data;
(iv) transmitting, to the first server the coordinator input data, wherein the coordinator input data comprises (A) the unique patient identifier associated with the first custom webpage, (B) the unique coordinator identifier associated with the particular coordinator computing device and (C) at least an amount of time the particular coordinator computing device interacted with each of the plurality of regions of the first custom webpage;
(c) the supervisor computing device, wherein the supervisor computing device is associated with a particular supervisor, and wherein the supervisor computing device is configured for:
(i) receiving, from the first server, the second information; and
(ii) displaying the second custom webpage.

18. The system of claim 17, wherein:
(a) the at least one processor of the first server is additionally configured for:
(i) generating third information, wherein the third information comprises a medical care plan corresponding with the uncontrolled medical attribute for the particular patient;
wherein the medical care plan comprises a plurality of cycles;
wherein each cycle includes a plurality of activities for the particular coordinator computing device to complete;
wherein each activity is completed upon receiving adequate coordinator input data into at least one of the plurality of regions of the first custom webpage; and,
wherein a second cycle cannot be started until all activities of a first cycle are completed;
(ii) transmitting, to the particular coordinator computing device, the third information, wherein the particular coordinator computing device is configured for displaying the third information on the first custom webpage proximate to the plurality of regions for receiving the coordinator input data acting as a guide for instructing coordinator; and
wherein the particular coordinator computing device is configured for displaying the third information on the first custom webpage.

19. The system of claim 17, wherein the system comprises a provider computing device associated with a provider, wherein:
(a) the at least one processor of the first server is additionally configured for:
(i) generating fourth information comprising (i) a patient identifier for the particular patient, (ii) and fourth custom webpage information for a fourth custom webpage;
(ii) transmitting the fourth information to the provider computing device;
(iii) receiving, from the provider computing device, provider input data;
(b) the provider computing device is configured for:
(i) receiving, from the first server, the fourth information;
(ii) displaying the fourth custom webpage;
(iii) receiving, from the provider, the provider input data, wherein the provider input data comprises (A) a unique provider identifier of the provider associated with a trigger event, (B) the unique patient identifier of the particular patient, and (C) second region information associated with where the trigger event occurred.
